(19) United States
(12) Patent Application Publication
(10) Pub. No.: US 2016/0175089 A1
(43) Pub. Date: Jun. 23, 2016

(54) CORRECTION OF FIRST RAY DEFORMITY VIA PERONEUS LONGUS TENDON MODIFICATION

(71) Applicant: First Ray, LLC, Logan, UT (US)

(72) Inventors: T. Wade Fallin, Hyde Park, UT (US); Robert W. Hoy, Essex Junction, VT (US)

(21) Appl. No.: 14/947,706

(22) Filed: Nov. 20, 2015

Related U.S. Application Data
(60) Provisional application No. 62/095,334, filed on Dec. 22, 2014.

(51) Int. Cl. A61F 2/08

(52) U.S. Cl. CPC A61F 2/0811

(57) ABSTRACT

Implants and techniques for correcting deformity of the first ray of a human foot are presented. The correction includes modifying the peroneus longus tendon.

### CROSS-REFERENCE TO RELATED APPLICATIONS

This application claims the benefit of U.S. Provisional Application No. 62/095,334, filed Dec. 22, 2014.

### FIELD OF THE INVENTION

The invention relates to methods, implants, and instruments for correcting first ray deformity by modifying the peroneus longus tendon.

### BACKGROUND

Various conditions may affect skeletal joints such as the elongation, shortening, or rupture of soft tissues associated with the joint and consequent laxity, pain, and/or deformity. Repairs of the soft tissues of joints such as those found in the human foot have been difficult. Effective, long lasting correction of deformities of the first ray of the human foot are needed.

### SUMMARY

The present invention provides methods, implants, and instruments for correcting first ray deformity by modifying the peroneus longus tendon.

In one example of the invention, a method of correcting a deformity of the first ray of the human foot includes shortening the peroneus longus tendon.

In another example of the invention, a method includes relocating the insertion of the peroneus longus tendon.

In another example of the invention, a method includes altering the path of the peroneus longus tendon.

In another example of the invention, a spacer includes an artificial cuboid notch insertable in the path of the peroneus longus tendon.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is side elevation view of a foot illustrating anatomic reference planes and relative directions;

FIG. 2 is a lateral view of a foot illustrating dorsiflexion and plantar flexion;

FIG. 3 is a coronal view of a foot illustrating inversion and eversion;

FIG. 4 is a dorsal view illustrating bones, tendons, and ligaments of the foot;

FIG. 5 is a plantar view illustrating bones, tendons, and ligaments of the foot;

FIG. 6 is a perspective view illustrating bones, tendons, and ligaments of the foot;

FIG. 7 is a medial view of the MTP joint of the first ray of the foot;

FIG. 8 is a sectional view taken along line 8-8 of FIG. 7;

FIG. 9 is a dorsal view of the MTC joint of the first ray of the foot;

FIG. 10 is a medial view of the MTC joint of the first ray of the foot;

FIG. 11 is a dorsal view illustrating deformity of the foot;

FIG. 12 is a plantar view illustrating deformity of the foot;

FIG. 13 is a sectional view similar to that of FIG. 8 but illustrating deformity of the foot;

FIGS. 14 and 15 are dorsal and plantar views of a human foot illustrating an angular deformity of the foot;

FIG. 16 is a plantar lateral view of a foot illustrating foot anatomy and an illustrative implant according to the present invention;

FIG. 17 is a plantar view of the foot illustrating foot anatomy and the implant of FIG. 16;

FIG. 18 is a cross sectional view of the foot illustrating foot anatomy and the implant of FIG. 16;

FIG. 19 is a perspective view of the implant of FIG. 16;

FIG. 20 is a front elevation view of the implant of FIG. 16;

FIG. 21 is a side elevation view of the implant of FIG. 16; and

FIG. 22 is a top plan view of the implant of FIG. 16.

### DESCRIPTION OF THE ILLUSTRATIVE EXAMPLES

The following illustrative examples describe implants, instruments and techniques for treating deformity of the first ray of the human foot. In particular, they describe ways of treating metatarsus primus varus by modifying the peroneus longus tendon.

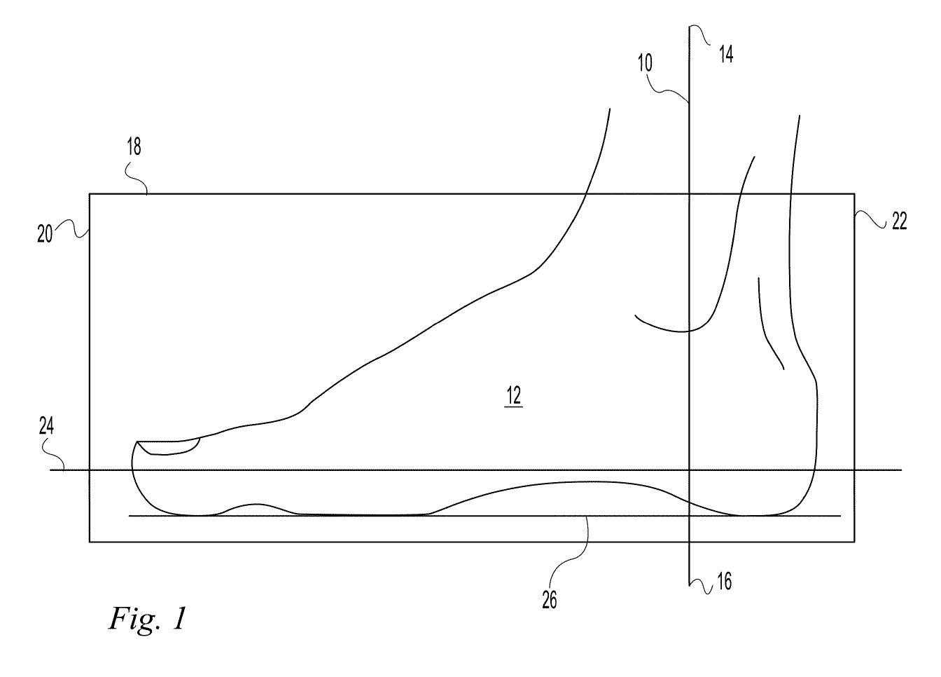

FIG. 1 illustrates the orientation of anatomic planes and relative directional terms that are used for reference in this application. The coronal plane 10 extends from medial 12 (toward the midline of the body) to lateral (away from the midline of the body) and from dorsal 14 (toward the top of the foot) to plantar 16 (toward the sole of the foot). The sagittal plane 18 extends from anterior 20 (toward the front of the body) to posterior 22 (toward the back of the body) and from dorsal 14 to plantar 16. The transverse plane 24 extends anterior 20 to posterior 22 and medial to lateral parallel to the floor 26. Relative positions are also described as being proximal or distal where proximal is along the lower extremity toward the knee and distal is along the lower extremity toward the toes. The following examples serve to demonstrate the relative directions. The great toe is medial of the lesser toes and the fifth toe is lateral of the great toe. The toes are distal to the heel and the ankle is proximal to the toes. The instep is dorsal and the arch is plantar. The toenails are dorsal and distal on the toes.

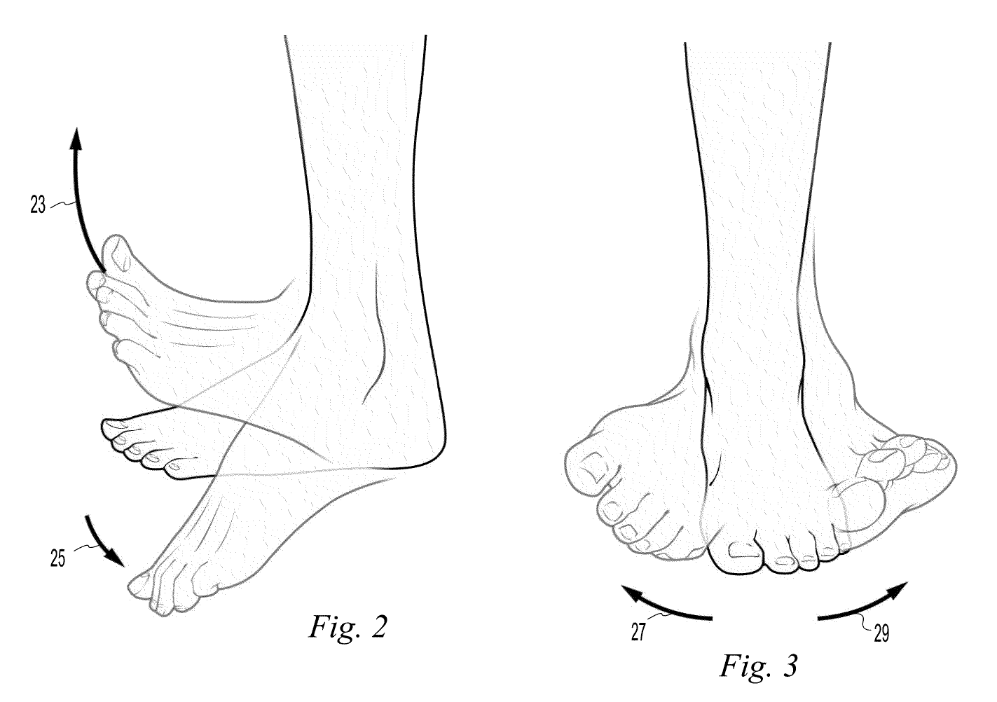

FIG. 2 illustrates dorsiflexion 23 in which the toes are moved dorsally, or closer to the shin, by decreasing the angle between the dorsum of the foot and the leg and plantar flexion 25 in which the toes are moved plantar, or further away from the shin, by increasing the angle between the dorsum of the foot and the leg. For example when one walks on their heels, the ankle is dorsiflexed and when one walks on their toes, the ankle is plantar flexed.

FIG. 3 illustrates inversion 27 in which the sole of the foot is tilted toward the sagittal plane or midline of the body and eversion 29 in which the sole of the foot is tilted away from the sagittal plane.

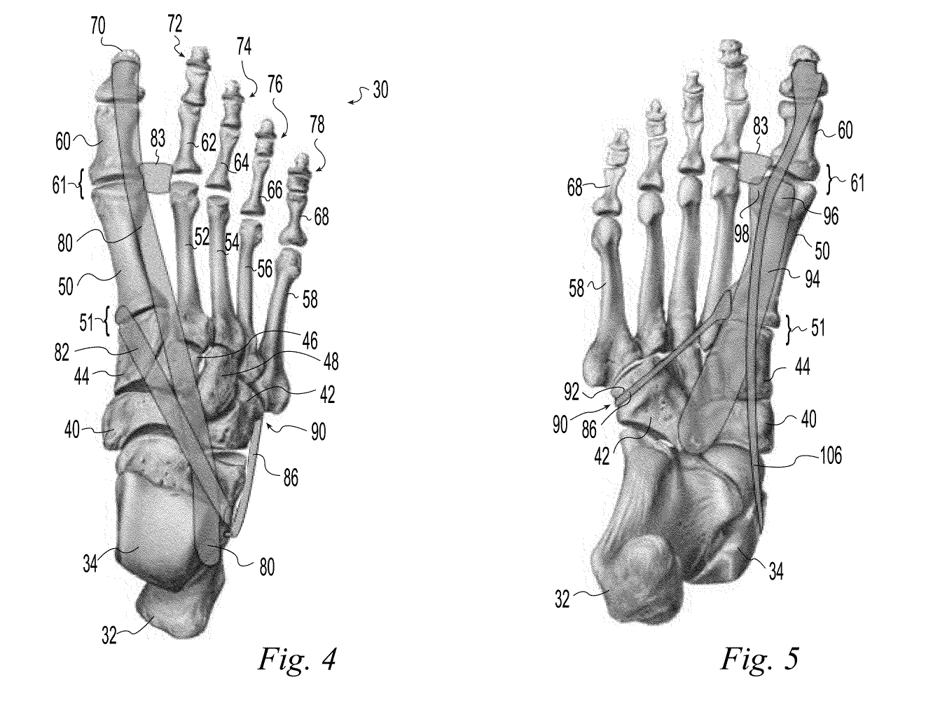

FIGS. 4-10 illustrate the arrangement of the bones within the foot 30. A right foot is illustrated. Beginning at the proximal aspect of the foot, the heel bone or calcaneus 32 projects plantar. The talus 34 is dorsal to the calcaneus 32 and articulates with it at the talocalcaneal or subtalar joint. Dorsally, the talus articulates medially with the tibia 36 and laterally with the fibula 38 at the ankle joint. Distal to the ankle are the navicular bone 40 medially and the cuboid bone 42 laterally which articulate with the talus and calcaneus respectively. The navicular bone 40 and cuboid bone 42 may also articulate with one another at the lateral side of the navicular bone and the medial side of the cuboid bone. Three cuneiform bones lie distal to the navicular bone and articulate with the navicular bone and one another. The first, or medial, cuneiform 44 is located on the medial side of the foot 30. The second, or intermediate, cuneiform 46 is located lateral of the first cuneiform 44. The third, or lateral, cuneiform 48 is located lateral of the second cuneiform 46. The third cuneiform 48 also articulates with the cuboid bone 42. Five metatarsals 50, 52, 54, 56, 58 extend distally from and articulate with the cuneiform and cuboid bones. The metatarsals are numbered from 1 to 5 starting with the first metatarsal 50 on the medial side of the foot and ending with the fifth metatarsal 58 on the lateral side of the foot 30. The first metatarsal 50 articulates with the first cuneiform 44 at a metatarsocuneiform (MTC) joint 51. The second metatarsal 52 articulates with the first, second and third cuneiforms 44, 46, 48 and may articulate with the first metatarsal as well. Five proximal phalanges 60, 62, 64, 66, 68 extend distally from and articulate with the five metatarsals respectively. The first proximal phalanx 60 articulates with the first metatarsal 50 at a metatarsophalangeal (MTP) joint 61. One or more distal phalanges 70, 72, 74, 76, 78 extend distally from the proximal phalanges. The first metatarsal 50, first proximal phalanx 60, and, first distal phalanx 70 together are referred to as the first ray of the foot. Similarly, the metatarsal, proximal phalanx, and distal phalanges corresponding to the lesser digits are referred to as the second through fifth rays respectively.

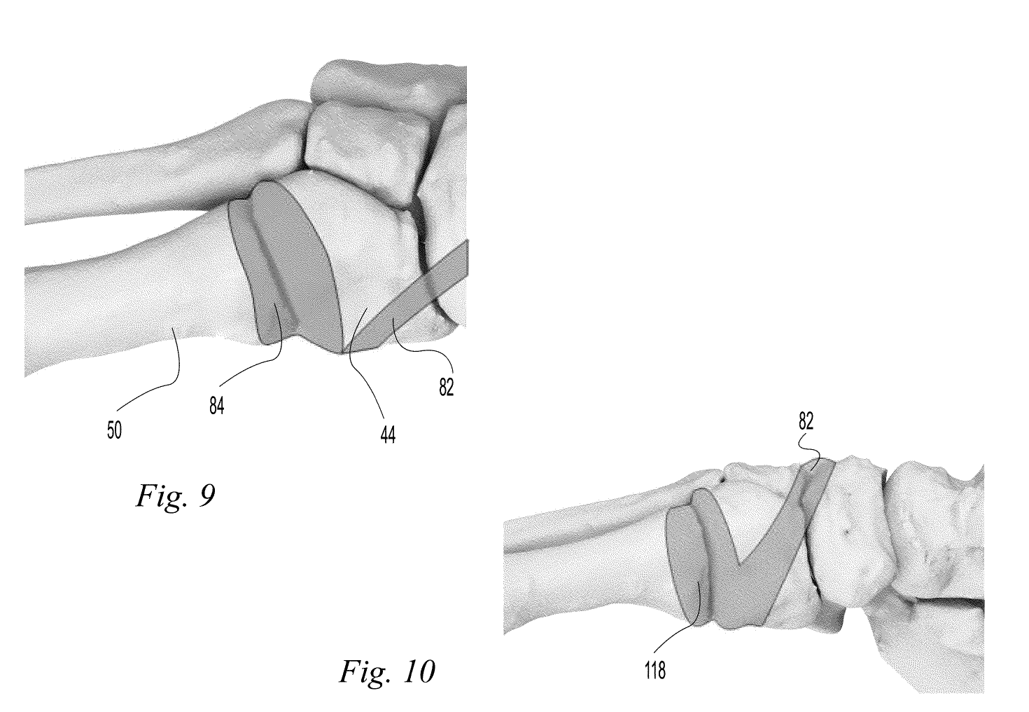

FIG. 4 is a dorsal view illustrating bones, tendons and ligaments of the foot. Plantar structures illustrated in FIG. 5 are omitted from FIG. 4 for clarity. The extensor hallucis longus muscle originates in the anterior portion of the leg, the extensor hallucis longus tendon 80 extends distally across the ankle and along the first ray to insert into the base of the distal phalanx 70. The tibialis anterior muscle originates in the lateral portion of the leg and the tibialis anterior tendon 82 extends distally across the ankle and inserts into the first cuneiform 44 and first metatarsus 50 at the first MTC joint 51 where it contributes to the MTC capsular structure 84 (FIGS. 9 and 10). A transverse intermetatarsal ligament 83 inserts into the capsule of the MTP joint such that it connects the heads of the first through fifth metatarsal bones. In FIG. 4, only the connection between the first and second metatarsal bones 50, 52 is shown.

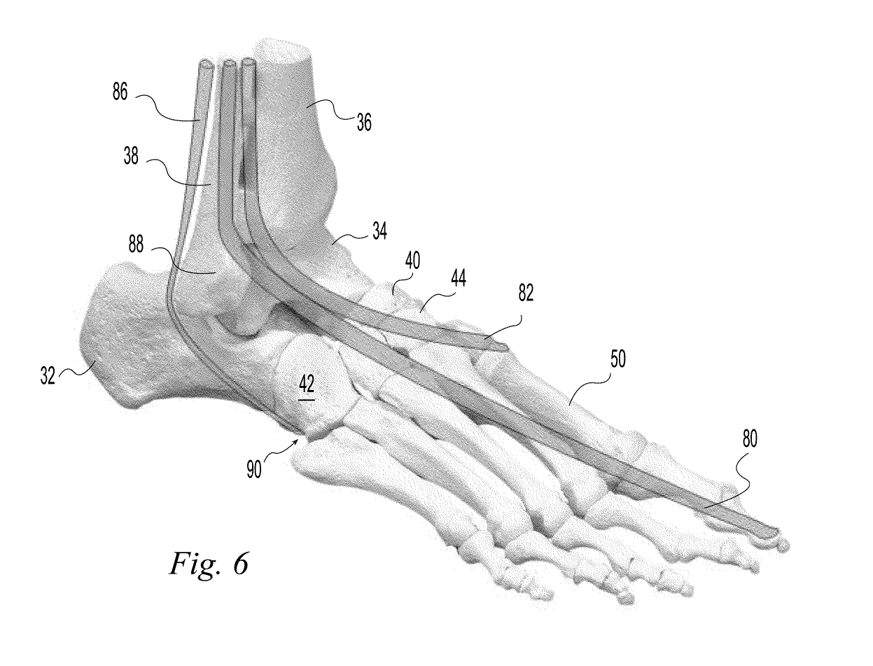

FIG. 5 is a plantar view illustrating bones, tendons, and ligaments of the foot. Dorsal structures shown in FIG. 4 are omitted from FIG. 5 for clarity. The peroneus longus muscle originates at the head of the fibula and its tendon 86 passes posteriorly around the lateral malleolus 88 (FIG. 6) of the ankle, around the cuboid notch 90 on the lateral side of the cuboid bone 42, along the peroneal sulcus 92 on the plantar surface of the cuboid bone 42, and inserts into the first metatarsal 50. The flexor hallucis brevis muscle 94 originates from the cuboid 42 and third cuneiform 48 and divides distally where it inserts into the base of the proximal phalanx 60. Medial and lateral sesamoid bones 96, 98 are present in each portion of the divided tendon at the MTP joint 61. The sesamoids 96, 98 articulate with the planar surface of the metatarsal head in two grooves 100, 102 separated by a rounded ridge, or crista 104 (FIG. 8). The flexor hallucis longus muscle originates from the posterior portion of the fibula 38. The flexor hallucis longus tendon 106 crosses the posterior surface of the lower end of the tibia, the posterior surface of the talus, runs forward between the two heads of the flexor hallucis brevis 94, and is inserted into the base of the distal phalanx 70 of the great toe.

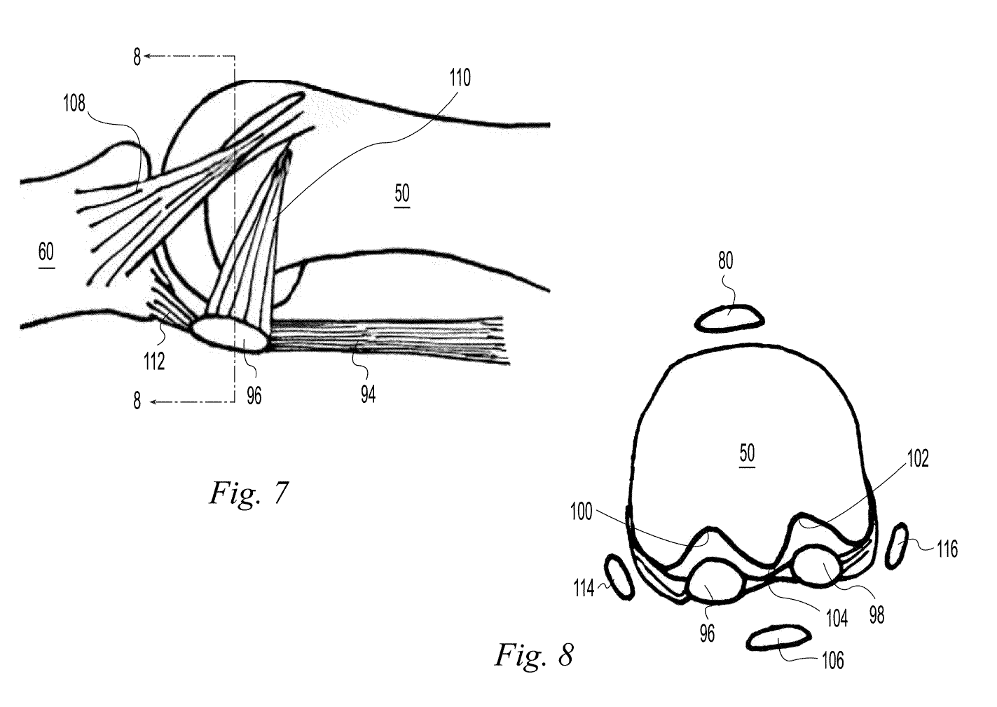

FIG. 7 is a medial view of tendons at the MTP joint 61 of the first ray. A medial collateral ligament 108 originates from the head of the first metatarsus 50 and inserts into the proximal phalanx 60. A medial metatarsosesamoid ligament 110 originates from the head of the first metatarsus 50 and inserts into the medial sesamoid bone 96. Similar collateral and metatarsosesamoid ligaments are found on the lateral side of the first MTP joint. The flexor hallucis brevis 94 is shown inserting into the sesamoids 96, 98. Ligamentous fibers extend further distally in the form of a phalangealsesamoid ligament 112 from the sesamoids to the proximal phalanx 60.

FIG. 8 is a sectional view taken along line 8-8 of FIG. 7 showing the metatarsal head 50, the tendon of the extensor hallucis longus 80, the medial and lateral sesamoid bones 96, 98, the grooves 100, 102 in which the sesamoids articulate, the crista 104 separating the grooves, the flexor hallucis longus 106, the abductor hallucis 114, and the adductor hallucis 116.

FIG. 9 is a dorsal view showing the dorsal capsular structure 84 of the MTC joint 51 of the first ray including the insertion of the tibialis anterior tendon 82.

FIG. 10 is a medial view of the MTC joint 51 of the first ray showing the medial capsular structure 118.

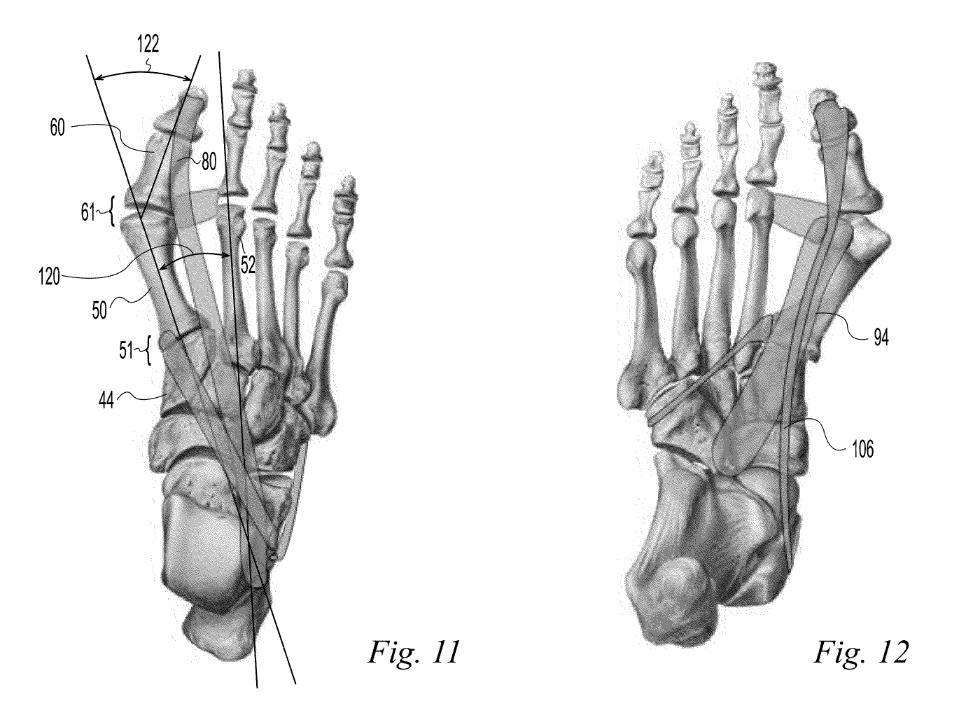

FIGS. 11-13 illustrate deformities of the first ray. In a dorsal view, as shown in FIG. 11, an intermetatarsal angle (IMA) 120 may be measured between the longitudinal axes of the first and second metatarsal bones 50, 52. The angle is considered abnormal when it is 9 degrees or greater and the condition is known as metatarsus primus varus (MPV) deformity. A mild deformity is less than 12 degrees, a moderate deformity is 12-15 degrees, and a severe deformity is greater than 15 degrees. Similarly, a hallux valgus angle (HVA) 122 may be measured between the longitudinal axes of the first metatarsus 50 and the first proximal phalanx 60 at the MTP joint 61. The angle is considered abnormal when it is 15 degrees or greater and the condition is known as a hallux valgus (HV) deformity. A mild deformity is less than 20 degrees, a moderate deformity is 20 to 40 degrees, and a severe deformity is greater than 40 degrees.

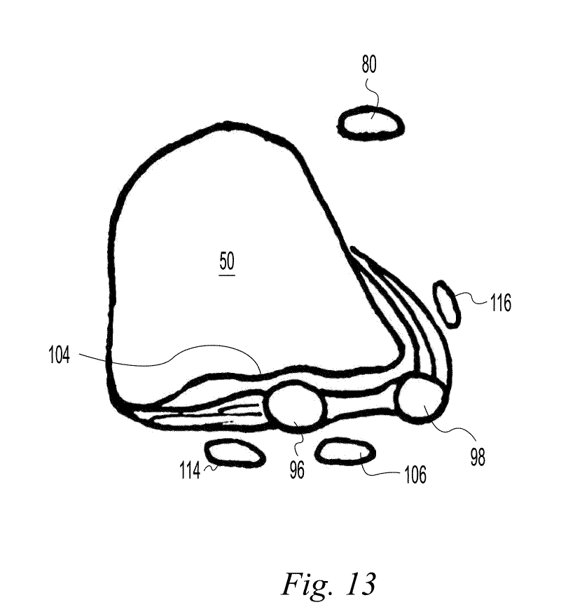

MPV and HV often occur together as shown in FIGS. 11-12. As the deformities progress several changes may occur in and around the MTC and MTP joints. Referring to FIG. 13, as the IMA and HVA increase, the extensors 80, flexors 106, abductors 114, and adductors 116 of the first ray (along with the sesamoids 96, 98) are shifted laterally relative to the MTP joint. The laterally shifted tendons exert tension lateral to the MTP joint creating a bow string effect (as best seen in FIGS. 11 and 12) that tends to cause the deformities to increase. The lateral shift of the sesamoids 96, 98 is often accompanied by erosion of the crista . The abnormal muscle forces cause the metatarsus 50 to pronate, or in other words, rotate so that the dorsal aspect of the bone moves medially and the plantar aspect moves laterally. Rotation in the opposite direction is referred to as supination. Soft tissues on the medial side of the MTP joint and lateral side of the MTC joint attenuate, through lengthening and thinning, thus weakening the capsule and permitting the deformities to progress. Soft tissues on the opposite sides of the capsule tend to shorten, thicken and form contractures making it difficult to reduce the joints to their normal angular alignment.

More generally, deformities of the first ray may include metatarsus primus varus, hallux valgus, abnormal pronation, abnormal supination , abnormal dorsiflexion, and/or abnormal plantarflexion. These deformities correspond to three different planar rotations. Metatarsus primus varus and hallux valgus result from rotations in the transverse plane 24. Pronation and supination are rotation in the coronal plane 10. Dorsiflexion and plantar flexion are rotation in the sagittal plane 20.

The terms “suture” and “suture strand” are used herein to mean any strand or flexible member, natural or synthetic, able to be passed through material and useful in a surgical procedure. The term “transverse” is used herein to mean crossing as in non-parallel.

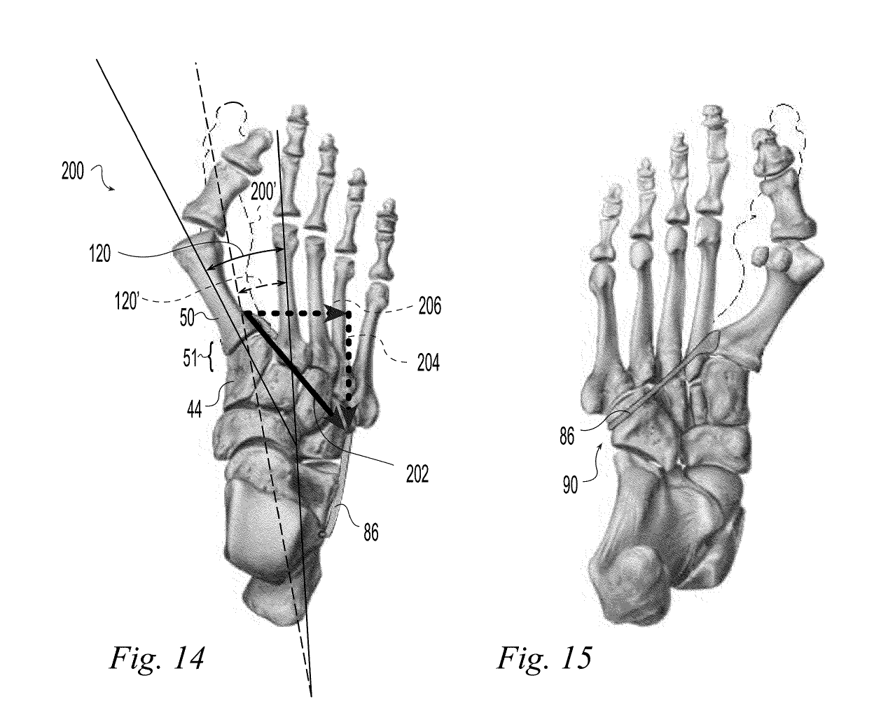

The present invention provides methods and devices for correcting first ray deformity by modifying the peroneus longus tendon 86 to produce a relative rotation of the first metatarsus 50 and cuneiform 44. FIGS. 14 and 15 illustrates the first ray of a right foot with an MPV deformity 200. The normal anatomic position 200′ of the first ray is shown in dashed lines. The peroneus longus tendon 86 inserts into the first metatarsus 50 just distal to the MTC joint 51 and exerts a force on the first metatarsus 50 as illustrated by force vector 202. This force vector 202 can be represented as the sum of component vectors 204 and 206 acting parallel and normal to the axis of the second metatarsus 52 from which the IMA 120 is referenced. The normal IMA 120′ is shown in dashed lines. The present investigators have found that since the peroneus longus tendon vector 202 acts close to the MTC joint, relatively small movements of the peroneus longus tendon result in relatively large rotations of the first metatarsus 50 in the transverse plane 24. For example, it has been found that a 1 mm movement of the peroneus longus tendon results in approximately 4 degrees of rotation of the first metatarsus and that a few millimeters of peroneus longus tendon movement can correct for example a 20 degree IMA to a normal IMA of approximately 8 degrees.

The peroneus longus tendon 86 may be modified in a variety of ways to correct an angular deformity. For example, the peroneus longus tendon may be shortened. For example, a portion of the tendon may be cut and the cut portions may be reattached in a shortened configuration. An example of a procedure of this type is a “Z”-plasty. The tendon may also be shortened by applying energy to the tendon to shrink the tendon. For example, heat or radio frequency energy may be applied to the tendon.

Another way of modifying the peroneus longus tendon 86 to correct an angular deformity is to detach the tendon at a connection to a bone and reattach the tendon at a different location on the bone.

Another way of modifying the peroneus longus tendon 86 to correct an angular deformity is to alter the path of the tendon 86. For example the path of the tendon 86 may be changed to effectively shorten the tendon 86 causing the tendon 86 to exert the lateral force on the first metatarsus 50 so that the first metatarsus 50 rotates about the first cuneiform 44 at the MTC joint 51 and reduces the intermetatarsal angle 120.

For example, the path may be changed by inserting a spacer between the peroneus longus tendon 86 and underlying tissue at some point along its path between the first metatarsus 50 and the leg. A convenient location for such a spacer is in or near the cuboid notch 90 of the cuboid bone. The cuboid notch 90 is located on the lateral side of the cuboid bone 42 and is readily accessible. Placing a spacer at this location will move the tendon 86 laterally effectively shortening it.

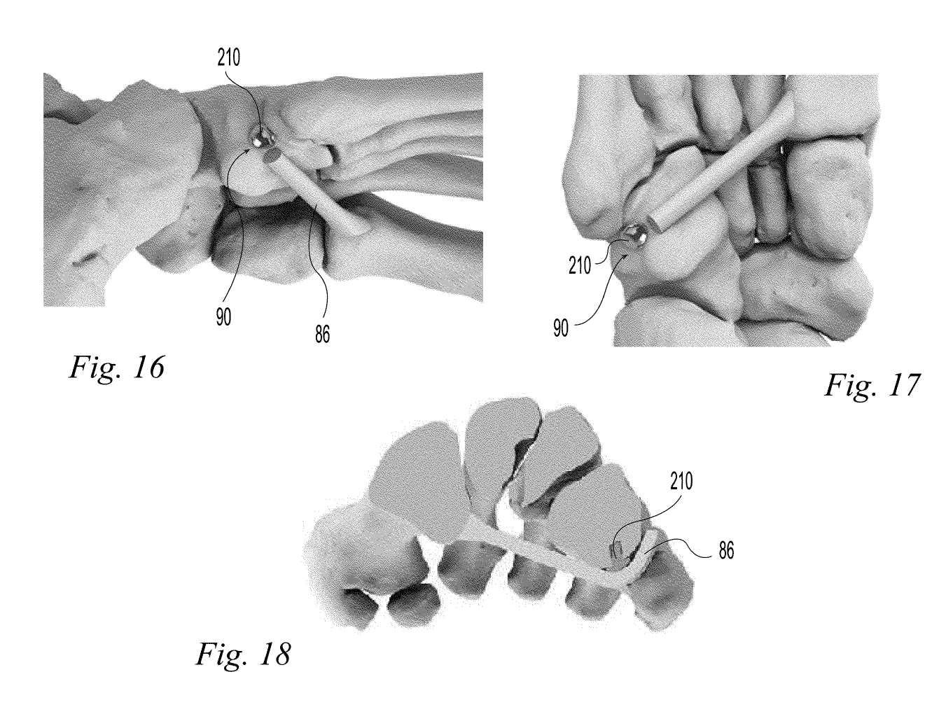

FIGS. 16-18 illustrate a spacer 210 placed in the cuboid notch 90. The peroneus longus tendon 86 is not fully shown so that the location of the spacer 210 may be better seen. As the tendon 86 travels over the spacer 210 its path is lengthened causing it to pull on the first metatarsus 50 and reduce the IMA.

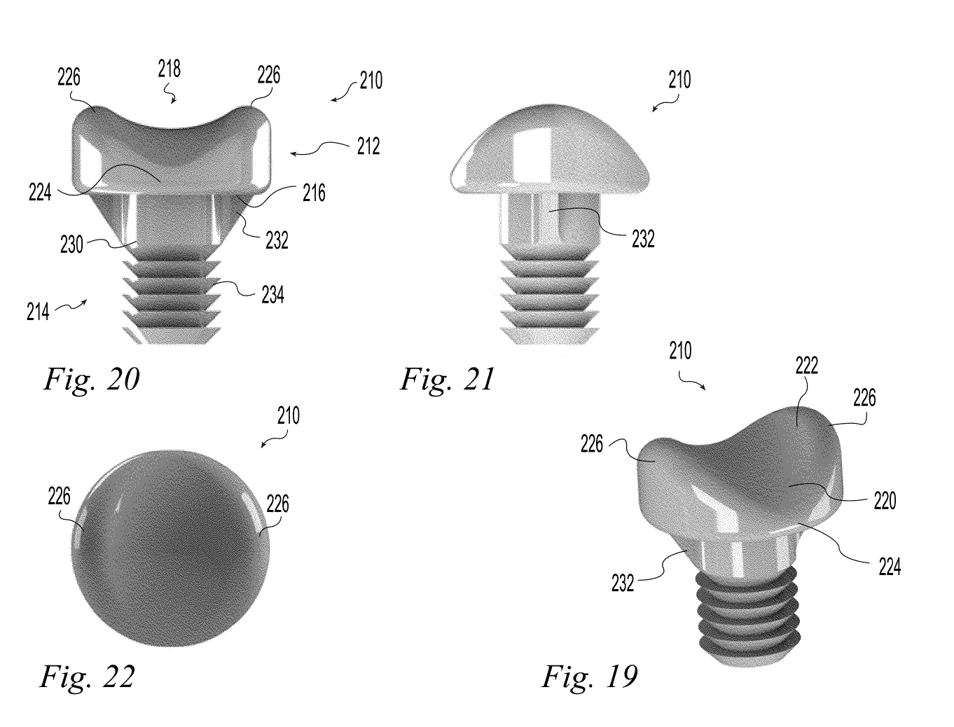

FIGS. 19-22 show the illustrative spacer 210 of FIGS. 16-18 in more detail. The spacer 210 includes a head 212 and an anchor portion 214. The head has a generally planar undersurface 216 and an opposite top surface defining a smooth bearing portion in the form of an artificial cuboid notch 218. The notch 218 is sized and shaped to receive the peroneus longus tendon 86 of the human foot in sliding relationship along the length of the tendon and restrict side-to-side motion, i.e. motion transverse to the length of the tendon, which could result in dislocation. The notch 218 has a saddle shape defined by a first, convex curve 220 toward the undersurface 216 in a first, longitudinal direction and a second, concave curve 222 away from the undersurface 216 in a second, transverse direction. The spacer defines a relatively thin edge 224 where the first curve 220 approaches the undersurface 216. The spacer defines raised side surfaces 226 where the second curve diverges from the undersurface 216.

The anchor portion 214 includes one or more projections extending from the undersurface 216 of the head opposite the artificial cuboid notch 218. In the illustrative example of FIGS. 19-22 the one or more projections include a post 230 and fins 232. The post 230 and fins 232 may be inserted into a bone to secure the spacer 210 to the bone. The fins help prevent rotation of the spacer 210 relative to the bone and thus maintain the artificial cuboid notch 218 in a preferred orientation. In the illustrative example of FIGS. 19-22 the post 230 also includes ridges 234 to aid in gripping the side walls of a hole formed in a bone.

The head 212 of the spacer 210 is generally cylindrical and has a diameter in the range of 4 to 10 mm. The post 230 is also generally cylindrical and has a diameter in the range of 2-5 mm. The spacer 210 may be made from one or more materials including plastic, metal, ceramic, autograft tissue, allograft tissue, xenograft tissue or other suitable materials.

What is claimed is:

1. A method of correcting a deformity of the first ray of the human foot, the method comprising:
modifying the peroneus longus tendon to produce a relative rotation of the first metatarsus and first cuneiform.

2. The method of claim 1 wherein modifying the peroneus longus tendon comprises changing the path of the tendon.

3. The method of claim 2 wherein changing the path of the tendon comprises lengthening the path of the tendon.

4. The method of claim 3 comprising placing a spacer between the peroneus longus tendon and underlying tissue.

5. The method of claim 4 comprising placing a spacer in or adjacent the cuboid notch of the cuboid bone to lengthen the path of the tendon around the cuboid bone.

6. The method of claim 5 further comprising engaging an anchor portion of the spacer with the bone and engaging the peroneus longus tendon with a smooth bearing portion of the spacer.

7. The method of claim 6 wherein the smooth bearing portion comprises a notch operable to capture the tendon to permit longitudinal tendon motion but restrict side-to-side tendon motion.

8. The method of claim 7 wherein the anchor portion includes a post, the method further comprising inserting the post into a hole formed in the bone.

9. The method of claim 1 wherein modifying the peroneus longus tendon comprises placing a spacer in the anatomic cuboid notch to form a new notch spaced from the anatomic cuboid notch to lengthen the tendon path and thereby effectively shorten the tendon causing the tendon to exert a laterally directed force on the first metatarsus so that the first metatarsus rotates about the first cuneiform at the metatarsocuneiform joint and reduces an intermetatarsal angle.

10. The method of claim 1 wherein modifying the peroneus longus tendon comprises shortening the tendon.

11. The method of claim 10 wherein shortening the peroneus longus tendon comprises cutting a portion of the tendon and reattaching the cut portion in a shortened configuration.

12. The method of claim 10 wherein shortening the peroneus longus tendon comprises applying energy to the tendon to shrink the tendon.

13. The method of claim 1 wherein modifying the peroneus longus tendon comprises detaching the tendon at a connection to a bone and reattaching the tendon at a different location on the bone.

14. An implantable spacer having a first portion defining an artificial cuboid notch and a second portion defining an anchor, the artificial cuboid notch being sized and shaped to receive the peroneus longus tendon of the human foot in sliding relationship along the length of the tendon and restrict motion transverse to the length of the tendon.

15. The implantable spacer of claim 14 wherein the anchor includes a projection, opposite the artificial cuboid notch, receivable in an opening to secure the spacer to a bone.

16. The implantable spacer of claim 14 further comprising a head on which the artificial cuboid notch is formed, the head having a generally flat undersurface opposite the artificial cuboid notch, the artificial cuboid notch having a saddle shape defined by a first, convex curve toward the undersurface in a first, longitudinal direction and a second, concave curve away from the undersurface in a second, transverse direction, the spacer defining a relatively thin edge where the first curve approaches the undersurface and the spacer defining raised side surfaces where the second curve diverges from the undersurface.

17. The implantable spacer of claim 15 wherein the anchor portion comprises a post extending from the undersurface of the head opposite the artificial cuboid notch.

18. The implantable spacer of claim 15 further comprising at least one fin extending between the undersurface of the head and the post.

19. The implantable spacer of claim 16 wherein the head is generally cylindrical and has a diameter in the range of 4 to 10 mm.

20. A method of correcting a deformity of the first ray of the human foot, the method comprising:
placing a spacer defining an artificial cuboid notch adjacent a human foot bone,
redirecting the peroneus longus tendon through the artificial cuboid notch to lengthen the tendon path and thereby effectively shorten the tendon causing the tendon to exert a laterally directed force on the first metatarsus so that the first metatarsus rotates about the first cuneiform at the metatarsocuneiform joint and reduces an intermetatarsal angle.
